(19) United States
(12) Patent Application Publication
(10) Pub. No.: US 2022/0288116 A1
(43) Pub. Date: Sep. 15, 2022

(54) HUMECTANT AND/OR BUFFERED STARCH COMPOSITIONS AND USE IN ORAL CARE SUCH AS TREATING XEROSTOMIA

(71) Applicant: GreenMark Biomedical Inc., East Lansing, MI (US)

(72) Inventor: Steven BLOEMBERGEN, Okemos, MI (US)

(21) Appl. No.: 17/638,614

(22) PCT Filed: Nov. 19, 2020
(86) PCT No.: PCT/US2020/061204

§ 371 Date: Feb. 25, 2022

Related U.S. Application Data
(60) Provisional application No. 62/937,901, filed on Nov. 20, 2019.

(51) Int. Cl. A61K 33/42; A61K 31/047; A61K 9/00

(52) U.S. Cl. CPC A61K 33/42; A61K 31/047; A61K 9/0053

(57) ABSTRACT

A composition is made with starch and glycerol and/or erythritol and/or a phosphate. The starch may be in the form of particles. Adding a combination of glycerol and erythritol increases the ability of the starch particles to be loaded with water. Optionally, starch particles are made with a phosphate crosslinker. The composition may be used as an oral care agent, for example for the treatment of xerostomia, caries or plaque.

### CROSS-REFERENCE TO RELATED APPLICATIONS

This application is a non-provisional application of U.S. application Ser. No. 62/937,901, filed Nov. 20, 2019.

### BACKGROUND

Xerostomia (also called dry mouth) involves a decrease in the production of saliva that is uncomfortable and can also lead to a range of other oral health issues.

Xerostomia can make it difficult for a person to speak or consume food. Among other complications, xerostomia also reduces the pH of the oral cavity, which in turn can lead to dental caries and promote cavities. Xerostomia can be initiated by a number of causes, some of which include diabetes, Sjogren's syndrome, aging and the use of many commonly prescribed medications. Xerostomia is a growing problem as the world's population average age continues to increase.

Mild symptoms of xerostomia can be relieved to some extent by lifestyle changes such as limiting the use of alcohol and caffeine and increasing fluid intake. Aqueous electrolyte sprays and sprays containing oxygenated glycerol triesters are also available to temporarily help relieve discomfort from xerostomia symptoms. Xerostomia symptoms may also be relieved via medications including sialagogues. Since cavities are frequently associated with dry mouth, xerostomia treatment can also include enhanced dental care such as using toothpaste with fluoride or amorphous calcium phosphate and using varnishes over white spots.

### INTRODUCTION

The following paragraphs are intended to introduce the reader to one or more aspects of the invention and the detailed description to follow but not to limit or define any claimed invention.

This specification describes starch compositions that contain glycerol and/or erythritol and/or a phosphate. In some examples, the composition includes starch and, (1) erythritol; (2) glycerol and erythritol; (3) a phosphate and glycerol; or, (4) a phosphate and erythritol. Optionally, the starch can be in the form of particles that can be greater than or less than 1 micron in Z-average size, or they can be a cluster of starch particles. A starch particle may have one or more starch molecules. Optionally, a starch particle may be one or more of a regenerated starch particle, a crosslinked starch particle, a starch fragment, or cold water soluble starch. The glycerol and/or erythritol and/or phosphate can be added before, during or after forming starch particles. In some examples, a combination of glycerol and erythritol is used. In some examples, the starch composition includes a phosphate such as sodium trimetaphosphate (STMP) or a reaction product of a phosphate. In some examples, the starch composition is hydrated. Optionally, a combination of glycerol and erythritol is used. In at least some examples, this combination appears to have a synergistic effect that increases moisture retention by the starch particles.

The starch composition can be used in an oral care product. For example, the starch composition can be dispersed or dissolved in water and sprayed into the mouth. In some examples, the starch can stabilize an emulsion of an oil, for example an oxygenated glycerol triester, in water. Alternatively or additionally, the starch composition can be incorporated into a food, gum, candy or lozenge. In some examples, the starch composition is used in a solid form, i.e. not in a solution or dispersion, but contains water by way of sorption.

The starch composition, or an oral care product including the starch composition, can be used for the treatment of xerostomia. In some examples, the starch composition or an oral care product including the starch composition may also be used to inhibit or treat caries and/or dental plaque. A process of treating xerostomia, caries and/or dental plaque may include applying the starch composition to the oral cavity of a person or other animal.

### DETAILED DESCRIPTION

This specification describes a starch composition useful as an oral care agent, for example for the treatment of xerostomia, caries and/or dental plaque.

The production of starch particles is described, for example, in International Publication Number WO 00/69916, Biopolymer Nanoparticles; International Publication Number WO 2011/155979, Process for Preparing Stable Dispersions of Starch Particles;

and, International Publication Number WO 2019/191456, Phosphate Crosslinked Starch Nanoparticle and Dental Treatment. All of these publications are incorporated herein by this reference to them. In these examples, native starch is dissolved in water using heat and/or a chemical agent and/or shear forces. The dissolved starch, optionally in a thermoplastic melt phase, is regenerated into particles having a size generally less than 1 or 2 micrometers, for example by crosslinking. Although the term “nanoparticle” is sometimes used in these publications, in many examples the particles may be smaller or larger than the IUPAC threshold of 100 nm for a nanoparticle and therefore, in some countries, some examples of the starch particles might be referred to as microparticles or sub-micron particles. Other methods of making starch particles may also be used. The size of starch particles may be determined as the z-average size or z-average mean as determined by ISO 13321 or ISO 22412, alternatively called the harmonic intensity averaged particle diameter, as determined by dynamic light scattering.

A composition may have starch particles. For the purposes of this specification, the term “starch particles” will include cold water soluble starch (which in some examples can refer to starch wherein the particle is a single molecule) unless specified otherwise. The composition may have other compounds but is primarily starch or starch-based, meaning that starch has the highest dry weight of any single component of the composition. Optionally, the composition can be made with 50% or more, or 65% or more, starch by dry weight.

In the examples described further below, starch compositions are made that include glycerol and/or erythritol. Optionally, the starch compositions may also include a phosphate or polyphosphate, for example sodium trimetaphosphate. The phosphate may be physically entrapped and/or covalently reacted and/or crosslinked onto the starch polymer.

Glycerol is a solvent and can be used as a plasticizer in place of, or in addition to water in the production of starch particles, for example by way of the method described in International Publication Number WO 00/69916, Biopolymer Nanoparticle. Alternatively or additionally, glycerol can be added to starch particles after they are formed, for example by soaking the particles in a glycerol solution. Starch particles may be crosslinked hydrogels that swell when dispersed in water. The starch particles can therefore take up aqueous compositions. Optionally, the starch particles can be dried after being soaked in an aqueous glycerol solution to remove at least some of the water while leaving glycerol in the starch particles. Similarly, glycerol can be added to non-crosslinked starch particles including cold water soluble starch. Glycerol is used in many foods and generally regarded as safe for human consumption.

Erythritol is a sugar alcohol used as a calorie-free sweetener. It has the simplified formula 04H1004 and is also known by the IUPAC designation (2R, 3S)-Butane-1,2,3,4-tetrol. Erythritol cannot be metabolized by tooth bacteria and therefore does not cause tooth decay. On the contrary, erythritol may be protective against tooth decay since it has antibacterial effects against streptococci bacteria and can reduce dental plaque. Erythritol occurs naturally in some foods or can be produced industrially. It is excreted unchanged in urine and generally regarded as safe for human consumption. As described in the experimental examples further below, erythritol can be added to starch at least by way of soaking the starch in an aqueous solution of erythritol. Other low-calorie or calorie-free sweeteners can also be used, including Xylitol, tagatose, sucralose, Aspartame, Acesulfame potassium, Neotame, Stevia, saccharin, Yacon Syrup as well as low-calorie or calorie-free sweeteners in combination with natural and artificial sugars and sweeteners including honey, maple syrup, agave nectar, and raw sugar.

Glycerol is used as a humectant in some manufactured foods. Erythritol is not strongly hygroscopic and is assumed in food science not to dry out other ingredients. As demonstrated in the experimental examples described below, glycerol when added to starch materially enhanced the ability of the starch to retain water. Erythritol, in contrast, provided no material increase in the ability of the starch to retain water. But surprisingly when glycerol and erythritol are used together they appear to provide a synergistic combination wherein the humectant properties of a starch composition is increased beyond what would be predicted based on the results for glycerol and erythritol alone.

Optionally, starch particles may be made with a phosphate or polyphosphate crosslinker as described in International Publication Number WO 2019/191456, Phosphate Crosslinked Starch Nanoparticle and Dental Treatment. In particular, the crosslinker may be sodium trimetaphosphate (STMP). STMP is an inorganic polyphosphate salt of formula (NaPO₃)₃. It is crystalline with a cyclic structure. STMP is used as a starch-modifying agent in some foods and is generally safe for human consumption. It is slowly hydrolyzed in the body and recovered from urine as intact molecules.

In the context of a product for treating xerostomia, using a phosphate or polyphosphate crosslinker to make the starch particles can provide other benefits. For example, STMP is somewhat inefficient as a crosslinker and produces compounds with dangling phosphate groups in the finished particles that are available for further reaction with soluble calcium from saliva in the mouth. Calcium is only sparingly soluble, as borne out by the low Molar Solubility Factor (Ksp) of calcium phosphate and other inorganic calcium salts. However, as the low level of soluble calcium binds with phosphates within the starch particles, in response new soluble calcium is immediately released and available for further binding to phosphates within the starch particles, thus providing a constant source of enamel restoration.

One or more of the resulting phosphate compounds may function as a buffering agent. The addition of a buffering agent is useful because the pH of patients with xerostomia and/or caries tends to be low, which encourages the formation and growth of carious lesions that can lead to cavities. A buffering agent can counter this tendency.

The starch composition can alternatively or additionally have other buffering compounds added to them. There are several buffer systems in the body. The most important include: (1) phosphate buffer, (2) bicarbonate buffer (HCO₃⁻/CO₂), (3) hemoglobin buffer (in erythrocytes), (4) proteins, and (5) ammonium buffer. The phosphate buffer system operates in the internal fluids of cells. It consists of dihydrogen phosphate ions as the hydrogen ion donor (acid) and hydrogen phosphate ion as the ion acceptor (base). If additional hydroxide ions enter the cellular fluid, they are neutralized by the dihydrogen phosphate ion. The phosphate buffer consists of phosphoric acid (H₃PO₄) in equilibrium with dihydrogen phosphate ion (H₂PO₄⁻) and H⁻. The pKa for the phosphate buffer is 6.8, which allows this buffer to function within its optimal buffering range at physiological pH. Buffering compounds added to the particles can include, for example, STMP, sodium metaphosphate, sodium polyphosphates and sodium hexametaphosphate.

STMP can also be added to starch, for example as a crosslinker or after forming particles or cold water soluble starch, for its anti-caries properties. Phosphate anions can bind calcium more tightly than sodium and therefore exchange their sodium ions when calcium ions are present. Calcium and/or fluoride ions may be added during the formation of starch particles, as described in International Publication Number WO 2019/191456, Phosphate Crosslinked Starch Nanoparticle and Dental Treatment. Alternatively, calcium ions are present in saliva. Calcium phosphates appear to be involved in the formation of enamel or enamel substitutes. For example, U.S. Pat. No. 4,132,772 describes the use of STMP as an anti-caries agent. International Publication Number WO 2019/191456, Phosphate Crosslinked Starch Nanoparticle and Dental Treatment, describes the use of compounds created while using STMP as a crosslinker for remineralization of caries and treatment of dentinal hypersensitivity.

STMP can also be added to starch, optionally as a crosslinker when forming starch particles, for its humectant properties. STMP can be hydrous, containing six molecules of water of hydration and three metaphosphate units. STMP loses water on storage at 20° C. but does not become anhydrous until heated to 100° C.

Starch particles can be dispersed in water or cold water soluble starch can be dissolved in water. The solution/dispersion can be sprayed in the mouth to provide relief from xerostomia symptoms or other oral conditions. Alternatively, the starch composition can be incorporated into a solid carrier such as a lozenge, candy, gum or food, for example mints, chocolate or baked goods. The starch composition may be hydrated in advance or become hydrated when mixed with other ingredients used to make an aqueous product or solid carrier. The starch composition is loaded with water that is released slowly into the mouth as the starch is degraded by amylase. Any other agents in the composition, for example phosphates, glycerol or erythritol or other sweetener, are also released into the mouth as the starch is degraded in the mouth.

Alternatively or additionally, starch can be used to stabilize an oil in water emulsion as in a Pickering emulsion, The starch is added to a mixture of oil in water that is emulsified under high shear mixing. The oil phase may be, for example, an oily lipid such as an oxygenated glycerol triester. Oxygenated glycerol triesters are commercially available, for example, from Ashland under the VINCIENCE trade mark. VINCIENCE oxygenated glycerol triesters are made from oxygenated corn oil. In particular, the double bonds in long fatty acid side chains of the corn oil have been oxidized away. Optionally, starch particles can be emulsified in an oily lipid or an oily lipid can be otherwise added to starch. The starch and oil can be applied to the mouth to provide relief from xerostomia symptoms or other oral conditions.

### Examples

Aqueous solutions were made with glycerol and/or erythritol. Table 1 gives the relative weight of the ingredients used in samples 1-4. The glycerol and/or erythritol, if any, were added to 10 g of water in a scintillation vial and dissolved using a magnetic stirrer. Bioform(™) starch from EcoSynthetix Inc, (a generally regarded as safe grade of extruded starch having a particle size of less than 1 micron) was added after removing the stir bar, and the mixtures were shaken to dissolve or disperse the starch. Portions of the solutions/dispersions were then added to wide plastic containers similar to an evaporating dish. Water was removed by evaporation in a fume hood at room temperature. The resulting plastic containers containing the dry films were then transferred to a humidity chamber and maintained at 15% relative humidity (RH) until their mass stabilized. This mass was deemed to be the dry starting mass. The humidity in the chamber was then increased and the compositions re-weighed after their weight stabilized at various higher humidity values. The increase in weight of the composition was determined and used as an indicator of the humectant properties of the starch compositions.

TABLE 1

| | Mass in | Mass in | Mass in | Mass in |
|---|---|---|---|---|
| Ingredient | Sample 1 (g) | Sample 2 (g) | Sample 3 (g) | Sample 4 (g) |
| Starch | 5.56 | 3.89 | 3.89 | 3.89 |
| Glycerol | 0 | 1.50 | 0 | 0.75 |
| Erythritol | 0 | 0 | 1.50 | 0.75 |

TABLE 2

| Sample | mass at 15% RH | mass at 40% RH | mass at 60% RH | | mass at 85% RH | |
|---|---|---|---|---|---|---|
| 1 | 3.67 g | 3.76 g (+2.4%) | 3.93 g | (+7.1%) | 4.89 g | (+33%) |
| 2 | 5.46 g | 5.62 g (+2.9%) | 6.05 g | (+10.8%) | 7.80 g | (+43%) |
| 3 | 4.51 g | 4.62 g (+2.4%) | 4.82 g | (+6.9%) | 5.74 g | (+27.3)% |
| 4 | 4.55 g | 4.79 g (+5.3%) | 5.03 g | (+10.5%) | 6.71 g | (+47.4)% |

As indicated in Table 2, adding erythritol alone (Sample 3) produces very little change relative to starch alone (Sample 1). Glycerol (Sample 2) produces an increase in the humectant properties of the composition. Sample 4 had humectant properties higher than that of Sample 2. This was a surprising result since Sample 4 contains erythritol but only half of the glycerol of Sample 2. The combination of erythritol and glycerol appears to have a synergistic effect on the ability of the composition to be hydrated, i.e. loaded with water.

1. A composition comprising starch and (1) erythritol; or, (2) glycerol and erythritol; or, (3) a phosphate and glycerol; or, (4) a phosphate and erythritol.

2. The composition of claim 1 comprising glycerol and erythritol.

3. The composition of claim 1 comprising a phosphate.

4. The composition of claim 1 comprising starch particles.

5. A method of making a composition comprising forming starch particles and then adding glycerol and/or erythritol and/or a buffering agent to the starch particles.

6. The method of claim 5 comprising crosslinking the starch particles with a phosphate crosslinker.

7. (canceled)

8. (canceled)

9. (canceled)

10. A method of treating xerostomia comprising adding glycerol and/or erythritol and/or a phosphate to the oral cavity of a person.

11. The method of claim 10 comprising adding glycerol and erythritol to the oral cavity of the person.

12. The method of claim 11 comprising adding a phosphate to the oral cavity of the person.

13. (canceled)

14. (canceled)

15. (canceled)

16. The composition of claim 1 comprising water or an oily lipid.

17. The composition of claim 1 comprising a solid carrier.

18. The composition of claim 17 wherein the carrier is selected from the group consisting of gum, candy, a lozenge and food.

19. The method of claim 5 wherein the composition is dispersed or dissolved in water or an oily lipid.

20. The method of claim 5 wherein the composition is contained in a solid carrier.

21. The method of claim 20 wherein the carrier is selected from the group consisting of gum, candy, a lozenge and food.

22. The method of claim 10 wherein the glycerol and/or erythritol and/or a phosphate is carried by starch.

23. The method of claim 10 wherein the glycerol and/or erythritol and/or a phosphate is carried by starch particles.

24. The method of claim 10 wherein the composition is dispersed or dissolved in water or an oily lipid.

25. The method of claim 10 wherein the composition is contained in a solid carrier.

26. The method of claim 25 wherein the carrier is selected from the group consisting of gum, candy, a lozenge and food.
